(19) United States
(12) Patent Application Publication
(10) Pub. No.: US 2024/0008775 A1
(43) Pub. Date: Jan. 11, 2024

(54) ELECTRONIC DEVICE AND METHOD FOR OBTAINING BIOMETRIC INFORMATION

(71) Applicant: Samsung Electronics Co., Ltd., Suwon-si (KR)

(72) Inventor: Hyunjun JUNG, Suwon-si (KR)

(21) Appl. No.: 18/347,951

(22) Filed: Jul. 6, 2023

Related U.S. Application Data
(63) Continuation of application No. PCT/KR2023/009518, filed on Jul. 5, 2023.

(30) Foreign Application Priority Data
Jul. 8, 2022 (KR) 10-2022-0084523
Sep. 30, 2022 (KR) 10-2022-0125523

(51) Int. Cl. A61B 5/1455; A61B 5/117; A61B 5/00

(52) U.S. Cl. CPC A61B 5/14552; A61B 5/117; A61B 5/7221; A61B 5/7246; A61B 2562/0233

(57) ABSTRACT

An electronic device is provided. The electronic device includes at least one optical sensor, a memory, and at least one processor electrically connected to the at least one optical sensor and the memory. The at least one processor may radiate light onto a user's body part in a specified section. The at least one processor may control the at least one optical sensor to receive light reflected from the user's body part. The at least one processor may obtain a plurality of optical signals having different wavelengths from the received light in the specified section. The at least one processor may obtain similarity information by comparing similarities between at least two specified optical signals among the plurality of optical signals. The at least one processor may identify the specified section as an abnormal section.

### CROSS-REFERENCE TO RELATED APPLICATION(S)

This application is a continuation application, claiming priority under § 365(c), of an International application No. PCT/KR2023/009518, filed on Jul. 5, 2023, which is based on and claims the benefit of a Korean patent application number filed on Jul. 8, 2022, in the Korean Intellectual Property Office, and of a Korean patent application number 10-2022-0125523, filed on Sep. 30, 2022, in the Korean Intellectual Property Office, the disclosure of each of which is incorporated by reference herein in its entirety.

### TECHNICAL FIELD

The disclosure relates to an electronic device and a method for obtaining biometric information.

### BACKGROUND ART

Recently, electronic devices have been developed in various forms for user convenience and have been miniaturized so that users are able to conveniently carry them. In addition, there are increasing interests in health and technology for checking health conditions.

Accordingly, an electronic device may include a sensor for measuring biometric information of a user and is being developed in various forms so as to measure and utilize various biometric signals of a human body using sensors, thereby providing various services for managing a user's health or checking a user's health status through a measurement of various biometric signals.

The above information is presented as background information only to assist with an understanding of the disclosure. No determination has been made, and no assertion is made, as to whether any of the above might be applicable as prior art with regard to the disclosure.

### DETAILED DESCRIPTION OF THE INVENTION

### Technical Solution

Aspects of the disclosure are to address at least the above-mentioned problems and/or disadvantages and to provide at least the advantages described below. Accordingly, an aspect of the disclosure is to provide an electronic method and a method for obtaining biometric information with high accuracy through pulse oximetry using a reference signal (e.g., an optical signal of a green or blue wavelength) having a high correlation with arterial blood.

Additional aspects will be set forth in part in the description which follows and, in part, will be apparent from the description, or may be learned by practice of the presented embodiments.

In accordance with an aspect of the disclosure, an electronic device is provided. The electronic device includes at least one optical sensor, a memory, and at least one processor electrically connected to the at least one optical sensor and the memory. The at least one processor may radiate light onto a user's body part in a specified section. The at least one processor may control the at least one optical sensor to receive light reflected from the user's body part. The at least one processor may obtain a plurality of optical signals having different wavelengths from the received light in the specified section. The at least one processor may obtain similarity information by comparing similarities between at least two specified optical signals among the plurality of optical signals. The at least one processor may identify that a venous pulsation is detected in the specified section, based on the similarity information. The at least one processor may identify the specified section as an abnormal section so as not to obtain biometric information using the plurality of optical signals in response to identifying that the venous pulsation is detected.

In accordance with another aspect of the disclosure, an operation method in an electronic device is provided. The operation method includes radiating light onto a user's body part and receiving light reflected from the user's body part in a specified section by at least one optical sensor of the electronic device. The method includes obtaining a plurality of optical signals having different wavelengths from the received light in the specified section. The method includes obtaining similarity information by comparing similarities between at least two specified optical signals among the plurality of optical signals. The method includes identifying the specified section as an abnormal section so as not to obtain biometric information using the plurality of optical signals in response to identifying that a venous pulsation is detected in the specified section, based on the similarity information.

In accordance with another aspect of the disclosure, a non-transitory storage medium is provided. The non-transitory storage medium may store a program that includes executable instructions configured to, when executed by a processor 120 of an electronic device 101, cause the electronic device 101 to radiate light onto a user's body part and receive at least some of light reflected from the user's body part in a specified section by at least one optical sensor 201 of the electronic device 101, obtain a plurality of optical signals 601, 602, and 603 having different wavelengths from the received light in the specified section, obtain similarity information by comparing similarities between at least two specified optical signals among the plurality of optical signals, and identify the specified section as an abnormal section so as not to obtain biometric information using the plurality of optical signals in response to identifying that a venous pulsation is detected in the specified section, based on the similarity information.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to various embodiments of the disclosure;

FIG. 2 is a block diagram illustrating a configuration of an electronic device according to an embodiment of the disclosure;

FIG. 3 is a block diagram illustrating an optical sensor of an electronic device according to an embodiment of the disclosure;

FIGS. 4A and 4B are diagrams illustrating obtaining of a plurality of optical signals in an optical sensor of an electronic device according to an embodiment of the disclosure;

FIGS. 5A and 5B are diagrams illustrating examples of a plurality of optical signals detected by an optical sensor of an electronic device according to an embodiment of the disclosure;

FIG. 6A is a graph illustrating a plurality of optical signals detected in a normal section according to an embodiment of the disclosure;

FIG. 6B is a diagram illustrating an example for identifying similarities between a plurality of optical signals in an abnormal section according to an embodiment of the disclosure;

FIG. 6C is a graph illustrating a plurality of optical signals detected in an abnormal section according to an embodiment of the disclosure;

FIG. 6D is a diagram illustrating an example for identifying similarities between a plurality of optical signals in a normal section according to an embodiment of the disclosure;

FIG. 7 is a graph illustrating an example of biometric information obtained from a normal section and an abnormal section in an electronic device according to an embodiment of the disclosure;

FIG. 8 is a flowchart illustrating an operation method of an electronic device according to an embodiment of the disclosure; and

FIG. 9 is a flowchart illustrating an operation method in an electronic device according to an embodiment of the disclosure.

Throughout the drawings, like reference numerals will be understood to refer to like parts, components, and structures.

### MODE FOR CARRYING OUT THE INVENTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

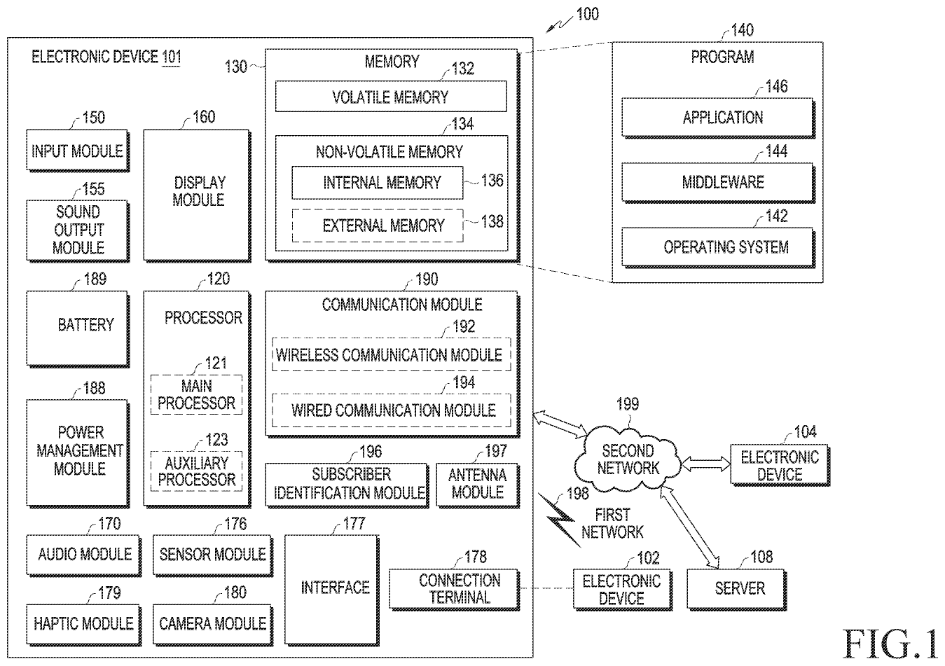

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may be configured to execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, a communication processor (CP), and the like) that is operable independently from, or in conjunction with, the main processor 121. In an example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control, for example, at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active (e.g., executing an application) state. According to one embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence model is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may be configured to store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or an external electronic device (e.g., an electronic device 102 (e.g., a speaker or a headphone)) directly or wirelessly coupled with the electronic device 101.

The sensor module 176 may be configured to detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, an illuminance sensor, and the like.

The interface 177 may support one or more specified protocols to be used for the electronic device 101 to be coupled with the external electronic device (e.g., the electronic device 102) directly or wirelessly. According to an embodiment, the interface 177 may include, for example, a high definition multimedia interface (HDMI), a universal serial bus (USB) interface, a secure digital (SD) card interface, an audio interface, and the like.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave band)) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 gigabits per second (Gbps) or more) for implementing 1eMBB, loss coverage (e.g., 164 database (dB) or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the external electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

An electronic device (e.g., an electronic device 101 in FIG. 1) in the disclosure may include various sensors (e.g., an optical sensor (e.g., a photoplethysmography (PPG) sensor) and a motion sensor) for measuring biometric signals, and the various sensors may be used to measure various biometric information such as a heart rate (or a pulse rate), a blood oxygen saturation, stress, and blood pressure of the user. The electronic device may be implemented in various forms to measure a biometric signal. For example, the electronic device may be implemented in the form of a wearable device that may be worn on the body of the user and measure a biometric signal of a body part of the user. The electronic device may measure a variety of biometric information of the user using the signals (e.g., biometric signals) detected through the sensors. Biometric information described in the disclosure may be called health information or other terms.

The electronic device in the disclosure may non-invasively measure the blood oxygen saturation among the biometric signals from a body part of the user by utilizing pulse oximetry, and the measurement may be performed using a ratio of absorbance of the increase blood flow between two wavelengths (e.g., a red wavelength and an infrared wavelength) using a temporary change in the volume of the arterial blood caused by the cardiac output. Biometric signal measurement utilizing the pulse oximetry may be made by measuring a ratio of hemoglobin (e.g., a value of about 90% or more in normal cases) combined with oxygen to the total hemoglobin concentration. The pulse oximetry is based on the assumption that only the arterial blood is the source of the periodic waveform measured. In addition, the pulse oximetry may be classified into a reflective structure type pulse oximetry and a penetrative type pulse oximetry. The reflective structure type pulse oximetry may be greatly affected by venous pulsation due to detection of a lot of blood flow in the venous bed under the skin, compared to the penetrative type pulse oximetry targeting arteries positioned deep inside the tissue. If the venous pulsation is included in a detected biometric signal, the accuracy of the measured biometric signal may be lowered. The venous blood is blood of low oxygen saturation that remains after oxygenating the tissues and may have a different phase (e.g., the opposite phase) from the arterial blood. The oxygen saturation of venous blood may offset a relatively high oxygen saturation value of the arterial blood, thereby reducing the measurement accuracy of the biometric signals. A complex calculation may be required to remove the venous pulsation and increase the measurement accuracy.

The disclosure provides an electronic device and a method for obtaining biometric information of high accuracy utilizing the pulse oximetry.

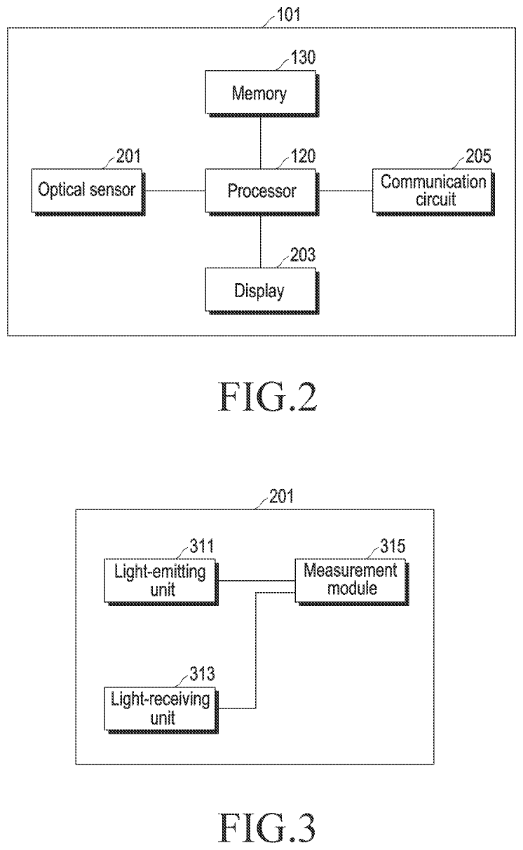

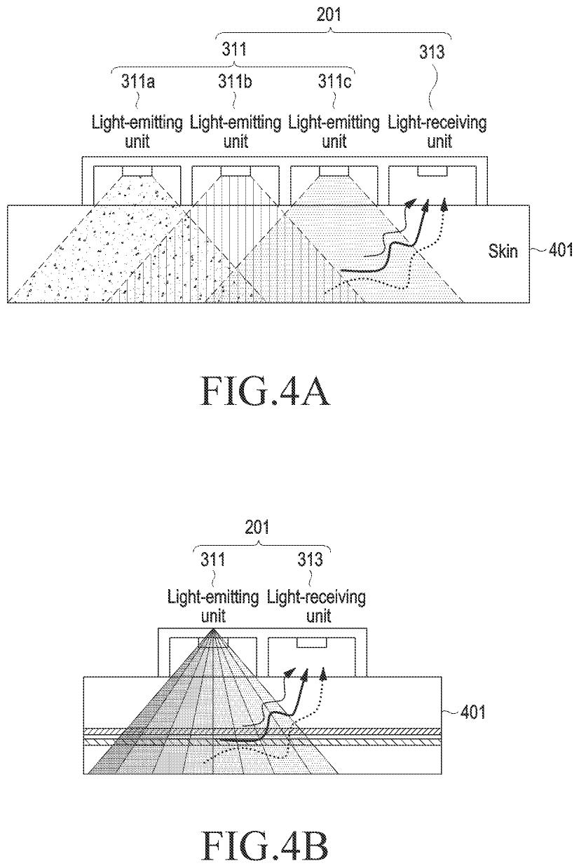

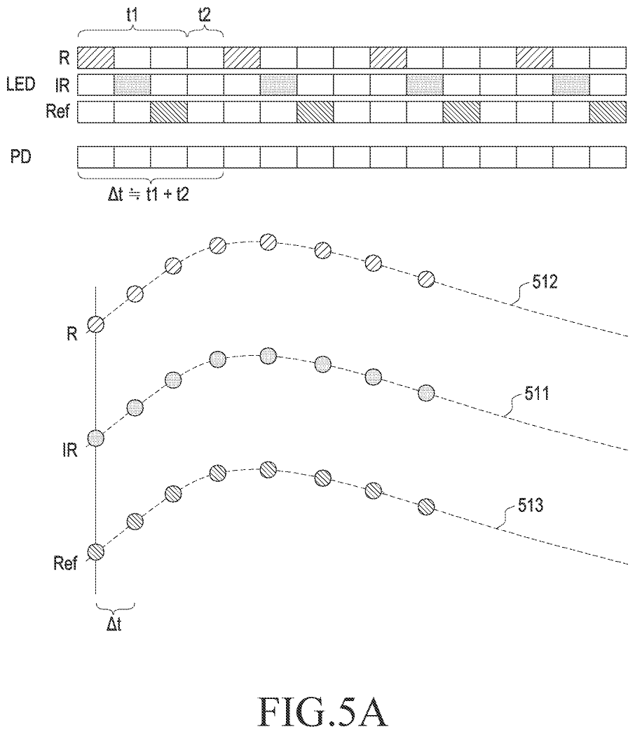

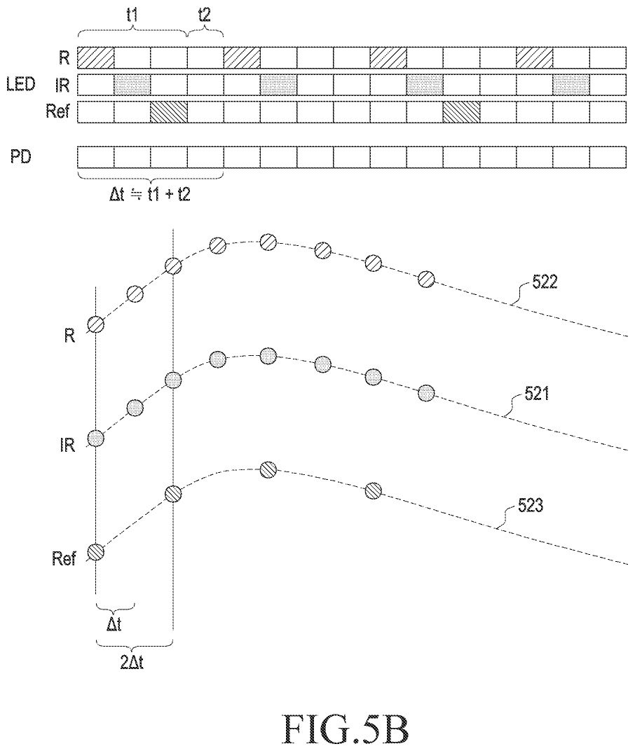

FIG. 2 is a block diagram illustrating the configuration of an electronic device according to an embodiment of the disclosure, and FIG. 3 is a block diagram illustrating an optical sensor of an electronic device according to an embodiment of the disclosure. FIGS. 4A and 4B are diagrams illustrating obtaining of a plurality of optical signals in an optical sensor of an electronic device according to an embodiment of the disclosure, and FIGS. 5A and 5B are diagrams illustrating examples of a plurality of optical signals detected by an optical sensor of an electronic device according to an embodiment of the disclosure.

Referring to FIGS. 1, 2, and 3, an electronic device 101 according to an embodiment may include at least one optical sensor 201 (e.g., the at least one optical sensor included in a sensor module 176 in FIG. 1), at least one processor 120, a memory 130, a display 203 (e.g., a display module 160 in FIG. 1 or a display included in the display module 160) and a communication circuit 205 (e.g., a communication module 190 in FIG. 1 or a communication circuit included in the communication module 190). The electronic device 101 is not limited thereto and may be configured by further including various elements or excluding some of the above elements. The electronic device 101 according to an embodiment may further include all or a part of the electronic device 101 shown in FIG. 1. In an example, the electronic device 101 may be implemented as a wearable device in a glasses type, a watch type, a patch type, a ring type, or other various types.

At least one optical sensor 201 according to an embodiment may detect a photoplethysmogram (PPG) signal for non-invasively measuring the oxygen saturation in a user's body part utilizing pulse oximetry, and the PPG signal (e.g., an optical signal) is a signal obtained by irradiating the tissues and the blood vessels with light and measuring reflected or penetrated light using a photodetector, which may be used to measure a change in the blood flow by a pulse wave.

Referring to FIGS. 1, 2, 3, 4A, and 4B, at least one optical sensor 201 according to an embodiment may include a light-emitting unit (e.g., a light-emitting element) 311, a light-receiving unit (e.g., a light-receiving element) 313, and a measurement module (e.g., a measurement element) 315. The configurations included in at least one optical sensor 201 are not limited to the light-emitting unit and the light-receiving unit. At least one optical sensor 201 may further include a signal processing unit (not shown) (e.g., an analog front end). For example, the signal processing unit (not shown) may include an amplifier for amplifying a biometric signal and an analog-to-digital converter (ADC) for converting an analog biometric signal to a digital biometric signal. However, the configurations included in the signal processing unit are not limited to the aforementioned amplifier and ADC.

According to an embodiment, at least one optical sensor 201 may be a photoplethysmography (PPG) sensor, and a change in the blood volume in the blood vessel may be measured by measuring the amount of reflected light using the optical sensor, based on a feature in which the volume of the blood vessel changes due to a change in the blood flow volume in the peripheral blood vessel as the heart repeats systole and diastole. The optical sensor 201 may include a light-emitting diode (LED), a laser diode (LD), an image sensor, or various types of sensors that output light to the outside or receive light from the outside.

According to an embodiment, at least one optical sensor 201 may output light to the outside through a plurality of light-emitting units 311 (311a, 311b, and 311c) shown in FIG. 4A or a single light-emitting unit 311 shown in FIG. 4B. The output light may be radiated onto the user's body, and at least some of the radiated light may be reflected by a user's body part 401 (e.g., skin, skin tissue, fat layer, vein, artery, or capillary blood vessel). At least one optical sensor 201, for example, may receive at least some of the light reflected by the user's body part 401 through the light-receiving unit 313 and output an electrical signal (hereinafter, a biometric signal) corresponding to the received light to at least one hardware element (e.g., a processor 120) of the electronic device 101.

According to an embodiment, at least one optical sensor 201 may output light to the outside through the light-emitting unit 311. In an example, the light-emitting unit 311 may output an infrared (IR) ray and visible light (e.g., red light, blue light, and/or green light) and include each light-emitting element (e.g., LED) corresponding to at least one output light.

According to an embodiment, at least one optical sensor 201 may configure at least one array. According to an embodiment, if there is a plurality of optical sensors, different weights may be applied to biometric signals obtained from the plurality of optical sensors. According to an embodiment, the optical sensor 201 may be disposed on a housing of a wearable device 200 or disposed to be exposed to the outside through the housing.

According to an embodiment, the light-emitting unit 311 of at least one optical sensor 201 may convert electrical energy into light energy. The light output from the light-emitting unit 311, for example, may include an infrared (IR) ray and visible light (e.g., red light, blue light, and/or green light). If light is transferred from the light-emitting unit 311 onto the skin, some of the light may be absorbed by the skin, and at least some of the remaining reflected light may be detected by at least one light-receiving unit 313. In the state in which at least one optical sensor 201 is in contact with the body, the amount of light detected through the light-receiving unit 313 during the systole of the heart may be reduced due to increased blood in the blood vessel, and the amount of light detected through the light-receiving unit 313 during the diastole of the heart may be increased due to reduced blood in the blood vessel. The measurement module 315 according to an embodiment may process a signal based on the amount of reflected light detected through the light-receiving unit 313, thereby measuring a variety of biometric information such as blood pressure, blood sugar, a heart rate, and a blood volume. For example, the light-emitting unit 311 may include at least one light-emitting element among a spectrometer, a vertical cavity surface emitting laser (VCSEL), a light-emitting diode (LED), a white LED, or a white laser. The light-emitting unit 311 may output IR light and/or visible light (e.g., red light, green light, or blue light) through the spectrometer, the VCSEL, the light-emitting diode (LED), the white LED, or the white laser.

According to an embodiment, the light-receiving unit 313 may receive (detect or sense) at least some of the light that is radiated by the light-emitting unit 311 and reflected from a user's body part 401 as shown in FIG. 4A or 4B. For example, the light-receiving unit 313 may convert light energy sensed by at least one light-receiving element into electrical energy. The light-receiving unit 313 may detect a first optical signal (e.g., a red optical signal) in a first wavelength band, a second optical signal (e.g., an IR optical signal) in a second wavelength band, and a third optical signal (e.g., a green optical signal) in a third wavelength band. The first wavelength and the second wavelength may be longer than the third wavelength, and the longer wavelength may be more sensitive to motion than the shorter wavelength. In another example, a noise component according to motion may be included in an optical signal sensed according to the motion during a light sensing operation, and if light sensing is performed in the same motion, a noise component according to the motion may be included more in the first optical signal (e.g., a red optical signal) than in the green optical signal. The light-receiving unit 313 may include at least one light-receiving element. In an example, the light-receiving unit 313 may include at least one of an avalanche photodiode (PD), a single-photon avalanche diode (SPAD), a photodiode, a photomultiplier tube (PMT), a charge coupled device (CCD), a CMOS array, or a spectrometer. In another example, the light-receiving unit 313 may have a reflective or penetrative structure. According to an embodiment, if an infrared ray (IR) and visible light (e.g., red light, blue light, and/or green light) are output from a single light-emitting unit 311 as shown in FIG. 4B, the light-receiving unit 313 may include at least one light-receiving filter (not shown) for selecting a desired wavelength band. According to an embodiment, the measurement module 315 or an integrated chip (IC) may be electrically connected to the light-emitting unit 311, the light-receiving unit 313, and the processor 120. The measurement module 315 may measure a biometric signal (e.g., an optical signal due to photoplethysmography) based on an electrical signal corresponding to the light (e.g., the amount of reflected light) received by the light-receiving unit 313. The measurement module 315 according to an embodiment may obtain a first optical signal (e.g., an IR optical signal) based on an electrical signal corresponding to first light (e.g., the amount of reflected IR light) detected by the light-receiving unit 313 and a second optical signal (e.g., a red optical signal) based on an electrical signal corresponding to second light (e.g., the amount of reflected red light) and obtain a third optical signal (e.g., a green or blue optical signal), as a reference signal, based on an electrical signal corresponding to third light (e.g., the amount of reflected green or blue light) detected by the light-receiving unit 313. The measurement module 315 according to an embodiment may transmit a plurality of the received optical signals (an IR optical signal and/or a red optical signal and a green optical signal) having different wavelengths to the processor 120 or process the same by itself.

The processor 120 of the electronic device 101 according to an embodiment may control at least one optical sensor 201 such that the light-emitting unit 311 of at least one optical sensor 201 radiates light onto the user's body part 401, such that the light-receiving unit 313 of at least one optical sensor receives at least some of light reflected from the user's body part, and such that the measurement module 315 detects a plurality of optical signals. The plurality of obtained optical signals having different wavelengths, for example, may be reflective photoplethysmography signals resulting from the light radiated from the light-emitting unit 311 and reflected or scattered by the user's body part.

According to an embodiment, the processor 120 may obtain a plurality of optical signals in different wavelength bands from the measurement module 315 of at least one optical sensor 201. Referring to FIGS. 5A and 5B, the processor 120 may repeatedly obtain a plurality of optical signals in different wavelength bands at the time of a specified section for detecting a plurality of optical signals. According to an embodiment, Referring to FIG. 5B, the processor 120 may configure the frequency of obtaining the third optical signal (e.g., a reference signal), which is a reference signal, to be different from the frequency of obtaining the first optical signal (e.g., an IR optical signal) and the second optical signal (e.g., a red optical signal), among the plurality of optical signals, to be utilized when the current consumption or the storage space of the memory 130 is insufficient. A processor 120 may configure the time for detecting the first optical signal and the second optical signal as a period of sampling interval Δt, which is a specified section, and configure the time for detecting the third optical signal as a multiple (e.g., 2Δt) of the period of sampling interval Δt, which is a specified section.

According to an embodiment, the processor 120 may sample a plurality of optical signals obtained in every specified section, as shown in graphs 511, 512, and 513 shown in FIG. 5A. According to an embodiment, as shown in the graphs 521, 522, and 523 shown in FIG. 5B, the processor 120 may sample comparison target signals (e.g., the first optical signal IR and/or the second optical signal R) among a plurality of optical signals obtained in every specified section Δt and sample a reference signal ref among a plurality of optical signals obtained in every time double the specified section 2Δt. The plurality of optical signals, for example, may include a first optical signal in a first light (e.g., IR) wavelength band, a second optical signal in a second light (e.g., red R) wavelength band, and a third optical signal in a reference wavelength band (e.g., green or blue wavelength) (ref). The first optical signal may be a reflective photoplethysmography signal in an infrared radiation (IR) wavelength band. The second optical signal may be a reflective photoplethysmography signal in a second light wavelength band. The third optical signal, as a reference signal, may be a reflective photoplethysmography signal in a blue wavelength band or green wavelength band, reflected from the shallow region of the user's skin. The specified section is a sampling interval (Δt≈t1+t2), which may be a section including a time t1 for alternately turning on the first optical signal, the second optical signal, and the third optical signal output from the light-emitting unit 311 and a time t2 for turning off least one optical sensor. If the bandwidth of at least one optical sensor (e.g., a PPG sensor) is about 10 Hz and if the sampling frequency is about 25 Hz, the sampling interval Δt may be configured as about 40 ms, and t1 may be configured as a short time of about a few hundred las. In this case, as shown in FIG. 5A, since the sampling intervals for LED wavelength bands (e.g., IR, R, and Ref) are much shorter than the bandwidth of a PD optical signal, it is possible to substantially simultaneously obtain optical signals in different wavelength bands. Since there are differences in the body absorption rates for the optical signals between wavelength bands (e.g., IR, R, and Ref) according to the optical signals output from the light-emitting unit 311, the processor 120 may control the light (LED) to be alternately turned on for respective wavelength bands (e.g., IR, R, and Ref), thereby configuring an on-section (section of time t1) of the specified section, so that the optical signal may be more precisely detected and the current consumption and the performance degradation may be reduced during long-term usage. In an embodiment, the processor 120 may configure a sampling rate, based on the performance-consumption current, to perform control to receive specific light according to a specified time interval while controlling on/off of the light-receiving unit 313 when the optical signal is received by the light-receiving unit 313.

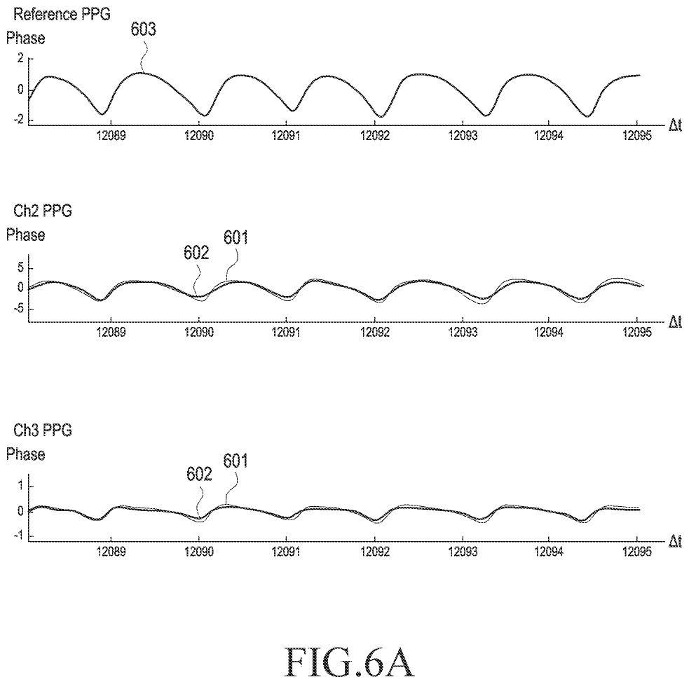

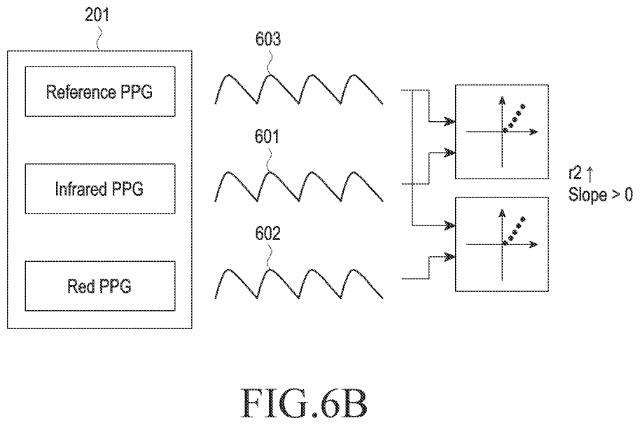

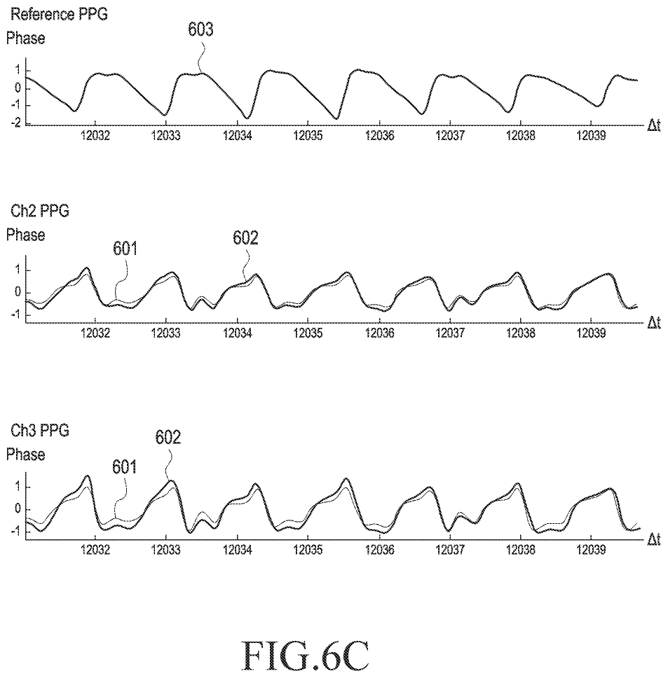

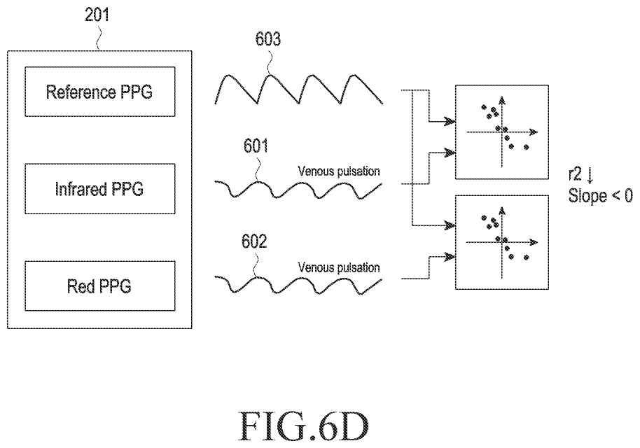

FIG. 6A is a graph illustrating a plurality of optical signals detected in a normal section according to an embodiment of the disclosure. FIG. 6B is a diagram illustrating an example for identifying similarities between a plurality of optical signals in an abnormal section according to an embodiment of the disclosure. FIG. 6C is a graph illustrating a plurality of optical signals detected in an abnormal section according to an embodiment of the disclosure. FIG. 6D is a diagram illustrating an example for identifying a similarities between a plurality of optical signals in a normal section according to an embodiment of the disclosure.

Referring to FIGS. 6A to 6D, according to an embodiment, a processor (e.g., a processor 120 in FIG. 2) may control an on/off operation of at least one optical sensor (e.g., LED) corresponding to each of a first optical signal 601, a second optical signal 602, and a third optical signal 603 to simultaneously obtain the first optical signal 601, the second optical signal 602, and the third optical signal 603 having different wavelengths in the specified section. The processor 120 according to an embodiment may compare similarities between a plurality of optical signals 601, 602, and 603 (e.g., a Ref signal 603 and an IR signal 601, or a Ref signal 603 and an R signal 602), based on the plurality of optical signals 601, 602, and 603 obtained in the specified section Δt, thereby obtaining similarity information according to the comparison result. The processor 120 may compare phases of at least two specified optical signals 601 and 603 or 602 and 603 in the specified section Δt. According to an embodiment, the processor 120 may compare the phases between the third optical signal 603 and the first optical signal (e.g., an IR optical signal) 601 or compare the phases between the third optical signal 603 and the second optical signal (e.g., a red optical signal) 602, thereby obtaining similarity information. For example, the third optical signal 603 may be a reference optical signal and may substantially include only arterial pulsation. A window length for obtaining similarity information may correspond to the specified section Δt and may include at least one beat. The similarity information may include a correlation value (e.g., the same phase value or different phase values) between the first optical signal 601 or the second optical signal 602 and the third optical signal 603, and slope information. The window may be a section of data to be checked to determine whether or not the periodic optical signals (e.g., IR, R, and Ref signals) are similar in two wavelengths, and the window length may include one or more periods and may be configured as several seconds (s) for fast response. The beat may indicate a pattern that appears periodically in the optical signals (e.g., IR, R, and Ref signals). In another example, the beat may indicate optical signals between the ventricular systole at a first time and the ventricular systole at a second time subsequent thereto.

According to an embodiment of the disclosure, the processor 120 may determine whether or not venous pulsation is detected in the specified section, based on the similarity information. The processor 120 may identify whether or not venous pulsation is detected from a comparison target signal (e.g., the first optical signal 601 and/or the second optical signal 602) among at least two specified optical signals compared. According to an embodiment, the processor 120 may or may not obtain biometric information in the specified section Δt depending on whether or not the venous pulsation is detected. The processor 120 may identify venous pulsation is detected in some (e.g., the first optical signal 601 and/or the second optical signal 602) of a plurality of optical signals repeatedly detected at intervals of the specified section Δt by at least one optical sensor, thereby obtaining biometric information according to the identification result. The processor 120 may repeatedly perform the operation of obtaining biometric information at the time of the specified section until a specified event (e.g., detection of a user's termination request or a user's motion less than or equal to a specified value) occurs.

According to an embodiment, Referring to FIGS. 6A and 6B, the processor 120 may identify that the phase of the third optical signal 603 detected in the specified section Δt, which is the current detection section, is substantially the same as the phase of the first optical signal (e.g., an IR optical signal) 601 or the phase of the second optical signal (e.g., an R optical signal) 602, based on the similarity information. In an embodiment, the processor 120 may identify that the current specified section is a normal section according to identification of substantially the same phase and identify that venous pulsation is not included in the first optical signal 601 or the second optical signal 602. As shown in FIGS. 6A and 6B, since the phase of the first optical signal 501 or the second optical signal 502 is substantially the same as that of the third optical signal 503, the correlation value between the first optical signal 601 or the second optical signal 602 and the third optical signal 603 may be high, and the slope r2 indicating correlation may be a positive number (slope>0) as shown in the graphs in FIG. 6B.

According to an embodiment, Referring to FIGS. 6C and 6D, the processor 120, based on the similarity information, may identify that the phase of the third optical signal 603 detected in the specified section is substantially different from the phase of the first optical signal (e.g., an IR signal) 601 or the phase of the second optical signal 602 (e.g., opposite phase) and, when the substantially different phases are identified, identify that venous pulsation is partially included in the first optical signal 601 or the second optical signal 602. As shown in FIGS. 6C and 6D, the correlation value between the first optical signal 601 or the second optical signal 602 and the third optical signal 603 may be low, and the slope r2 indicating correlation may be a negative number (slope<0).

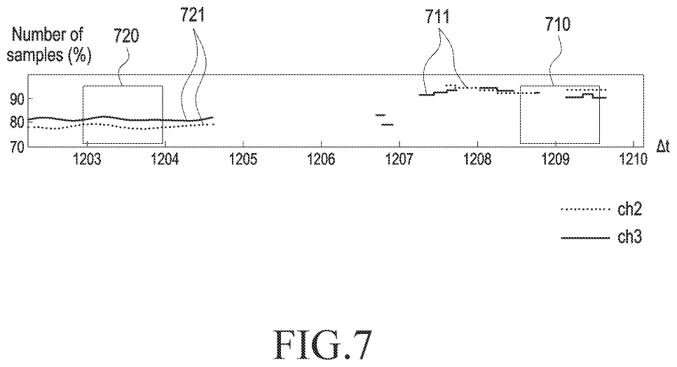

FIG. 7 is a graph illustrating an example of biometric information obtained from a normal section and an abnormal section in an electronic device according to an embodiment of the disclosure.

According to an embodiment, Referring to FIGS. 6A, 6B, and 7, if the correlation value included in the similarity information represents substantially the same phase, a processor (e.g., a processor 120 in FIG. 2) may identify that venous pulsation is not detected from some (e.g., the first optical signal and/or the second optical signal) of a plurality of optical signals. For example, if venous pulsation is not detected, the processor 120 may identify the detection section is a normal section 710 to obtain biometric information using the plurality of optical signals. The normal section 710 is a section in which only the arterial pulsations are substantially detected from some (e.g., the first optical signal and/or the second optical signal) of the plurality of optical signals, and biometric information with relatively high accuracy may be obtained using arterial pulsation. According to an embodiment, if venous pulsation is not detected, the processor 120 may obtain biometric information including high-oxygen saturation information (SpO2) 711 (e.g., the number of samples in ch2 and ch3 of about 95% or more) from the normal section 710 and control a display (e.g., the display 203 in FIG. 2) to display the obtained biometric information. The processor 120 may control a communication circuit (e.g., the communication circuit 205 in FIG. 2) to transmit the obtained biometric information to an external electronic device.

According to an embodiment, Referring to FIGS. 6B, 6D, and 7, if venous pulsation is detected, the processor 120 may identify the specified section as an abnormal section 720 so as not to obtain biometric information using the plurality of optical signals. The abnormal section 720 is a section in which the arterial pulsation and the venous pulsation are detected in some (e.g., the first optical signal and/or the second optical signal) of the plurality of optical signals, and since low-oxygen saturation information (SpO2) 721 (e.g., the number of samples in ch2 and ch3 of about 80% or less) is obtained in the abnormal section 720 due to the venous pulsation having a substantially different phase (e.g., the opposite phase) from that of the arterial pulsation, the accuracy of biometric information may be lowered. Accordingly, according to an embodiment, if the current section is identified as an abnormal section 720, the processor 120 may not perform the operation of obtaining oxygen saturation information or biometric information including the oxygen saturation information. According to an embodiment, the processor 120 may not output, to the display 203, or transmit, to an external electronic device (e.g., the electronic device 102, the electronic device 104, and/or the server 108 in FIG. 1), the biometric signal including the low-oxygen saturation information 721 obtained from the abnormal section 720. If the specified section is identified as the abnormal section 720, biometric information may not be obtained from the specified section.

According to an embodiment, the electronic device 101 may receive a plurality of optical signals in different wavelength bands in a plurality of regions (e.g., the channels ch2 and ch3 in FIGS. 6A, 6C, and 7) of the user's body using a plurality of optical sensors. The electronic device 101 may perform operations for obtaining the aforementioned biometric information for each of the plurality of regions by the processor 120. The plurality of regions of the body may be different regions adjacent to each other (e.g., regions spaced about 10 mm apart from each other).

According to an embodiment, if all the current sections are normal sections in the plurality of regions, the processor 120 of the electronic device 101 may obtain biometric information in each of the plurality of regions. According to an embodiment, if the current section is identified as a normal section in some of the plurality of regions and if the current section is identified as an abnormal section in other regions of the plurality of regions, the processor 120 of the electronic device 101 may obtain biometric information only in the region identified as the normal section. According to an embodiment, if the current section is identified as a normal section in some of the plurality of regions and if the current section is identified as an abnormal section in other regions of the plurality of regions, the processor 120 of the electronic device 101 may obtain biometric information in all of the plurality of regions. According to an embodiment, if the region of high priority (e.g., the region in which the ratio of being identified as a normal section during the previous specific time is high) among the plurality of regions is a normal section, and if other some regions (e.g., the region that is intermittently identified as a normal section for the previous specific time so that the ratio of being identified as the normal section is low) are abnormal sections, the processor 120 of the electronic device 101 may obtain biometric information from the region having the high priority. According to an embodiment, if all of the plurality of regions are identified as abnormal sections, the processor 120 of the electronic device 101 may not perform the operation of obtaining biometric information

According to an embodiment, if biometric information is obtained from the plurality of regions, the processor 120 of the electronic device 101 may obtain the biometric information from the respective regions simultaneously, in parallel, or sequentially.

According to an embodiment, although the electronic device 101 is able to obtain biometric information by directly detecting the user's body part, the electronic device 101 may obtain a plurality of optical signals having different wavelengths from another external electronic device through communication with the external electronic device. The electronic device 101 may identify whether or not venous pulsation is detected based on similarity information between at least two optical signals among the plurality of obtained optical signals and obtain biometric information according to whether or not the venous pulsation is detected.

According to an embodiment, the processor 120 may identify a similarity between at least two optical signals among the plurality of obtained optical signals while considering that the optical signals in respective wavelength bands have relatively small phase differences. The processor 120, for example, may compare the phases of the at least two specified optical signals, i.e., the first optical signal (IR) or the second optical signal (Red) until the time sequentially shifted to earlier (e.g., the time about 100 ms earlier) or delayed (e.g., the time about 100 ms later) by a specified time interval (e.g., 1 sample at a time, 4 ms based on 25 Hz) based on the reference signal, thereby identifying the similarity. The processor 120 may identify a similarity having a maximum value, among N (e.g., 100/4=25) similarity values obtained through the phase comparison until the early-shifted time or delayed time by a specified time interval as a final similarity for obtaining the venous pulsation, and may obtain similarity information including the final similarity.

The processor 120 of the electronic device 101 according to an embodiment may be a hardware module or a software module (e.g., an application program) and may be a hardware element (function) or software element (program) including at least one of various sensors, an input/output interface, a module managing the state or environment of the electronic device 101, or a communication module equipped in the electronic device 101.

According to an embodiment, the processor 120 may include, for example, one of hardware, software, or firmware, or a combination of two or more thereof. The processor 120 may be configured to exclude at least some of the above elements or further include other elements for performing the operation of obtaining biometric information in addition to the above elements. The processor 120 according to an embodiment may be configured as at least one or more processors, and the at least one processor may include a main processor performing high-performance processing and an auxiliary processor performing low-power processing, which are physically separate, and may be driven by the main processor and the auxiliary processor, respectively. The auxiliary processor may be connected to various biometric signal measurement sensors, thereby performing real-time (or 24-hour) monitoring of the biometric signals. According to an embodiment, a single processor 120 may operate, and a single processor may operate with high performance or perform low-power processing according to circumstances.

The processor 120 of the electronic device 101 according to an embodiment may intermittently perform an operation of identifying whether or not there is influence of venous pulsation on a plurality of optical signals detected by at least one optical sensor or an operation of obtaining biometric information. In an example, the processor 120 may obtain a reference signal (e.g., the third optical signal) at specified intervals (e.g., about 1 minute or about 10 minutes) and compare the same with other optical signals (e.g., the first optical signal or the second optical signal) obtained from the specified section continuously or at specified intervals. If there is no motion so that the same state is assumed to remain, the processor 120 may obtain biometric information only immediately after detecting the motion.

The memory 130 according to an embodiment may store information and/or data related to the operation of the electronic device 101. The memory 130 according to an embodiment may store instructions, when executed in the electronic device 101, causing the processor 120 to perform the above-described operation. In an example, the memory 130 may store an application (or program) related to the function of obtaining biometric information. In another example, the memory 130 may store information related to a plurality of optical signals having different wavelengths detected by at least one optical sensor, similarity information, information identifying whether or not venous pulsation is detected in a plurality of optical signals, and obtained biometric information. According to an embodiment, the memory 130 may store information on user motion detected by the motion sensor and information related to other biometric information detected using other sensors. The memory 130 according to an embodiment may store a variety of data generated during the execution of the program 140, as well as a program (e.g., the program 140 in FIG. 1) used for the function operation. The memory 130 may include a program area and the data area (not shown). The program area may store program information for driving the electronic device 201, such as the operating system (OS) (e.g., the operating system 142 in FIG. 1) for booting the electronic device 201. The data area (not shown) may store transmitted and/or received data and generated data according to various embodiments. Additionally, the memory 130 may be configured to include at least one storage medium of flash memory, a hard disk, multimedia card micro type memory (e.g., secure digital (SD) or extreme digital (XD) memory), RAM, and ROM.

In an embodiment, the display 203 (e.g., the display module 160 in FIG. 1) according to an embodiment may display a variety of information based on control of the processor 120. In the case where the display 203 according to an embodiment is implemented together with an input module (e.g., the input module 150 in FIG. 1 or an input circuit) in the form of a touch screen, it may display a variety of information produced according to a user's touch operation. According to an embodiment, the display included in the display 203 may be configured as at least one or more of a liquid crystal display (LCD), a thin film transistor LCD (TFT-LCD), an organic light-emitting diode (OLED), a light-emitting diode (LED), an active matrix organic LED (AMOLED), a micro LED, a mini LED, a flexible display, a 3-dimensional (3D) display, and the like. In addition, some of these displays may be of a transparent type or a light-transmission type such that the outside is able to be seen through them. This may be configured as a transparent display form including the transparent OLED (TOLED). According to an embodiment, the electronic device 101 may further include other display (e.g., an expandable display or a flexible display) mounted thereto in addition to the display 203.

According to an embodiment of the disclosure, the communication circuit 205 (e.g., the communication module 190 in FIG. 1) may communicate with an electronic device (e.g., the electronic device 102, the electronic device 104, and/or the server 108 in FIG. 1) of the outside (e.g., doctor or hospital), based on the control of the processor 120. Based on the control of the processor 120, the communication circuit 205 according to an embodiment may transmit obtained biometric information or transmit a plurality of optical signals having different wavelengths detected by at least one optical sensor to an external electronic device. The communication circuit 205 according to an embodiment may receive a plurality of optical signals having different wavelengths detected by at least one optical sensor in the external electronic device. For example, the communication circuit 205 may perform at least one of cellular communication, ultra-wide band (UWB) communication, Bluetooth communication, and/or wireless fidelity (WiFi) communication, and further perform other communication methods capable of communicating with the external electronic device.

The electronic device 101 according to an embodiment may further include a motion sensor (e.g., an accelerometer, a gyroscope, a barometer, and/or a geomagnetic sensor) for detecting the user's motion. The accelerometer, for example, may detect an acceleration or impact due to the motion of the electronic device 101 or the user possessing the electronic device 101. The gyroscope may detect a rotation direction or rotation angle of the electronic device 101 due to the motion of the electronic device 101 or the user possessing the electronic device 101. The barometer, for example, may detect the atmospheric pressure, and the geomagnetic sensor may detect the direction of the geomagnetic field. An operation (or motion) state of the user may be identified using accelerometric information, gyroscopic information, barometric information, and/or geomagnetic sensing information detected by the motion sensor according to an embodiment. The user's motion state may be identified as a state in which there is no motion (e.g., stationary), a state in which there is no motion or slight motion is detected (e.g., sedentary), or a state of motion (or a specified user activity state) (e.g., a walking state or a running state).

According to an embodiment, the electronic device 101 may further include at least one other sensor for detecting a biometric signal in addition to the optical sensor 201 and the motion sensor. For example, at least one other sensor (not shown) may include a body temperature sensor, an electrocardiogram (ECG) sensor, an electrodermal activity (EDA) sensor, and/or a sweat sensor. The body temperature sensor according to an embodiment may measure the body temperature. The ECG sensor according to an embodiment may measure the electrocardiogram by detecting an electrical signal from the heart through an electrode attached to the body. The EDA sensor according to an embodiment may include, for example, a galvanic skin response (GSR) sensor and measure the user's excited state by detecting the electrical activity on the skin. The sweat sensor according to an embodiment may detect sweat of the user's body to measure the hydration and/or dehydration. According to an embodiment, at least one biometric sensor may provide the processor 120 with a biometric signal measured by detecting a user's biometric signal, based on the control of the processor 120, and information (a value or a numerical value) (e.g., skin temperature, electrocardiogram, stress, skin conductivity, hydration, and/or dehydration) based on a biometric signal measured by detecting a user's biometric signal.

According to an embodiment, the electronic device 101 may further include an audio module (not shown) (e.g., an audio module 170 in FIG. 1) or a vibration module (not shown) (e.g., a haptic module 179 in FIG. 1). The audio module may output sound and may be configured to include, for example, at least one of an audio codec, a microphone (MIC), a receiver, an earphone output (EAR_L), or a speaker. The audio module, for example, may output an audio signal related to biometric information according to an embodiment, based on the control of the processor 120. The vibration module may output vibration related to biometric information according to an embodiment, based on the control of the processor 120.

As described above, the fundamental elements of an electronic device have been described through the electronic device 101 in FIGS. 1 and 2 in an embodiment. However, in various embodiments, not all of the elements shown in FIGS. 1 and 2 are essential elements, and the electronic device 101 may be implemented with more elements than the illustrated elements, or the electronic device 101 may be implemented with fewer elements. Additionally, the positions of the fundamental elements of the electronic device 101 described with reference to FIGS. 1 and 2 may vary according to various embodiments.

According to an embodiment, an electronic device (e.g., the electronic device 101 in FIGS. 1 and 2) may include at least one optical sensor (e.g., the optical sensor included in the sensor module 176 in FIG. 1 or the optical sensor 201 in FIG. 2), a memory (e.g., the memory 130 in FIGS. 1 and 2), and at least one processor (e.g., the processor 120 in FIGS. 1 and 2) electrically connected to the at least one optical sensor and the memory.

According to an embodiment, the at least one processor may radiate light onto a user's body part in a specified section.

According to an embodiment, the at least one processor may control the at least one optical sensor to receive light reflected from the user's body part.

According to an embodiment, the at least one processor may obtain a plurality of optical signals (e.g., the plurality of optical signals 601, 602, and 603 in FIGS. 6A to 6D) having different wavelengths from the light received in the specified section.

According to an embodiment, at least one processor may obtain similarity information by comparing similarities between at least two specified optical signals among the plurality of optical signals.

According to an embodiment, at least one processor may identify that a venous pulsation is detected in the specified section, based on the similarity information.

According to an embodiment, at least one processor may identify the specified section as an abnormal section so as not to obtain biometric information using the plurality of optical signals in response to identifying that the venous pulsation is detected.

According to an embodiment, at least one processor may be configured to not obtain the biometric information in the specified section in response to identifying the specified section as an abnormal section.

According to an embodiment, at least one processor may identify that the venous pulsation is not detected in the specified section, based on the similarity information.

According to an embodiment, at least one processor may identify the specified section as a normal section in response to identifying that the venous pulsation is not detected.

According to an embodiment, at least one processor may obtain the biometric signal in the specified section. The biometric information may include blood oxygen saturation information.

According to an embodiment, at least one processor may compare phases of the at least two specified optical signals among the plurality of optical signals. According to an embodiment, at least one processor may be configured to identify a similarity between the at least two specified optical signals, based on the comparison result.

According to an embodiment, at least one processor may based on identifying that the phases of the at least two specified optical signals are the same, identify that the venous pulsation is not detected from the at least two specified optical signals.

According to an embodiment, at least one processor may based on identifying that the phases of the at least two specified optical signals are different, identify that the venous pulsation is detected from some of the at least two specified optical signals.

According to an embodiment, at least one processor may compare the phase of at least one of the first optical signal or the second optical signal among the at least two specified optical signals with a phase of a reference signal until the time shifted to earlier or delayed by a specified time interval based on the reference signal among the at least two specified optical signals, and identify a similarity having a maximum value among similarity values obtained by the comparing the phase of at least one of the first optical signal or the second optical signal with the phase of the reference signal.

According to an embodiment, the at least two specified optical signals may include at least one of a first optical signal and a second optical signal to be compared and a third optical signal as a reference signal.

According to an embodiment, the first optical signal may be a reflective photoplethysmography signal having an infrared radiation (IR) wavelength.

According to an embodiment, the second optical signal may be a reflective photoplethysmography signal having a red wavelength.

According to an embodiment, the third optical signal may be a reflective photoplethysmography signal having a blue wavelength or green wavelength, reflected from the shallow region of the user's skin.

According to an embodiment, the specified section (e.g., the sampling interval Δt in FIG. 5A) (e.g., about 30 ms) may include a time t1 (e.g., about a few hundred μs) for alternately turning on the at least one optical sensor and a time t2 for turning off the at least one optical sensor.

According to an embodiment, the electronic device may further include a display electrically connected to the at least one processor. According to an embodiment, at least one processor may be configured to control the display to display the biometric information.

According to an embodiment, the electronic device may further include a communication circuit electrically connected to the at least one processor. According to an embodiment, at least one processor may be configured to control the communication circuit to transmit the biometric information to an external electronic device.

According to an embodiment, at least one processor may control the at least one optical sensor to obtain the plurality of optical signals in the specified section if a motion of the user is detected to be greater than or equal to a threshold value by at least one motion sensor.

According to an embodiment, at least one processor may control the at least one optical sensor not to obtain the plurality of optical signals in the specified section if the motion of the user is detected to be less than or equal to the threshold value by the at least one motion sensor.

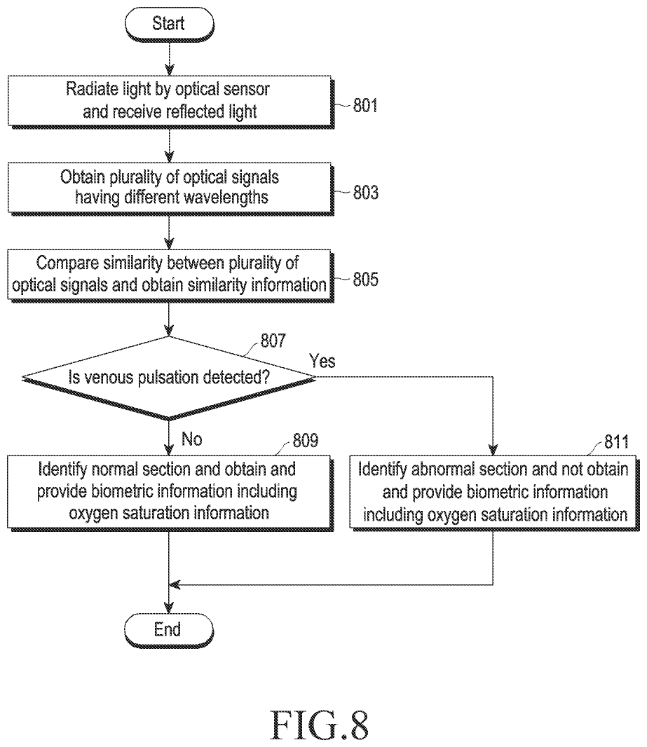

FIG. 8 is a flow chart illustrating an example of an operation method of an electronic device according to an embodiment of the disclosure.

Although respective operations may be sequentially performed in the following embodiment, the operations are not necessarily performed sequentially. In an example, the sequence of the respective operations may vary, and at least two operations may be performed in parallel.

According to an embodiment, operations 801 to 811 may be performed by a processor (e.g., a processor 120 in FIGS. 1 and 2) of an electronic device (e.g., an electronic device 101 in FIGS. 1 and 2).

Referring to FIG. 8, an electronic device (e.g., the electronic device 101 in FIGS. 1 and 2) according to an embodiment may radiate light onto a user's body part using at least one optical sensor in a specified section in operation 801. The electronic device may receive at least some of light reflected from the user's body part by at least one optical sensor.

In operation 803, the electronic device may obtain a plurality of optical signals having different wavelengths detected by at least one optical sensor in the specified section. The specified section may be a period of sampling interval Δt for obtaining a plurality of optical signals. The period of sampling interval Δt (e.g., about may be a section including a time t1 (e.g., about a few hundred μs) for alternately turning on at least one optical sensor and a time t2 for turning off at least one optical sensor.

In operation 805, the electronic device may compare a similarity between at least two optical signals among the plurality of optical signals, thereby obtaining similarity information according to the comparison result. According to an embodiment, the electronic device may compare a comparison target signal (e.g., a first optical signal 601 and/or a second optical signal 602 in FIGS. 6A to 6D) among the at least two specified optical signals with a reference signal (e.g., the third optical signal 603 in FIGS. 6A and 6D) among the at least two specified optical signals. The first optical signal may be a reflective photoplethysmography signal having an infrared radiation (IR) wavelength. The second optical signal, for example, may be a reflective photoplethysmography signal having a red wavelength. The third optical signal may be a reflective photoplethysmography signal having a blue wavelength or green wavelength, reflected from the shallow region of the user's skin. A window length for obtaining similarity information, for example, may correspond to the specified section and may include at least one beat. The similarity information may include a correlation value (e.g., the same phase value or different phase (e.g., opposite phase) values) between the first optical signal or the second optical signal and the third optical signal, and slope information. The window may be a section of data to be checked to determine whether or not the periodic optical signals (e.g., IR, R, and Ref signals) are similar in two wavelengths, and the window length may include one or more periods and may be configured as several seconds (s) for fast response. The beat may indicate a pattern that appears periodically in the optical signals (e.g., IR, R, and Ref signals). For example, the beat may indicate optical signals between the ventricular systole at a first time and the ventricular systole at a second time subsequent thereto.

In operation 807, the electronic device may identify whether or not venous pulsation is detected in the specified section, based on the similarity information. The electronic device may identify whether or not venous pulsation is detected from the comparison target signal (e.g., the first optical signal 601 or the second optical signal 602 in FIGS. 6A to 6D) among at least two specified optical signals compared. As a result of the identification, if venous pulsation is not detected, the electronic device may perform operation 809, and if venous pulsation is detected, the electronic device may perform operation 811.

In operation 809, the electronic device may obtain biometric information including oxygen saturation information (an SpO2 value) in response to non-detection of the venous pulsation. The electronic device, for example, may provide the obtained biometric information. The electronic device may display the obtained biometric information on the display (e.g., the display module 160 in FIG. 1 or the display 203 in FIG. 2) and/or transmit the same to an external electronic device (e.g., the electronic device 102, the electronic device 104, and/or the server 108 in FIG. 1) through the communication circuit (e.g., the communication module 190 in FIG. 1 or the communication circuit 205 in FIG. 2). According to an embodiment, if the correlation value included in the similarity information represents substantially the same phase, the electronic device may identify that venous pulsation is not detected from some (e.g., the first optical signal and/or the second optical signal) of a plurality of optical signals. If venous pulsation is not detected, the electronic device may identify the detection section is a normal section to obtain biometric information using the plurality of optical signals. The normal section is a section in which only the arterial pulsations are substantially detected from some (e.g., the first optical signal and/or the second optical signal) of the plurality of optical signals, and biometric information (e.g., an oxygen saturation value) with high accuracy may be obtained using arterial pulsation.

In operation 811, the electronic device may not perform the operation for obtaining biometric information including oxygen saturation information (an SpO2 value) in response to partial detection of the venous pulsation. According to an embodiment, if low-oxygen saturation information is identified, the electronic device may ignore the low-oxygen saturation information and may not provide biometric information. According to an embodiment, if biometric information includes information related to other biometric signals in addition to the oxygen saturation information, the electronic device may provide biometric information excluding the oxygen saturation information. According to an embodiment, if the correlation value included in the similarity information represents substantially different phases (e.g., opposite phases), the electronic device may identify that venous pulsation is detected from some (e.g., first optical signal and/or second optical signal) of the plurality of optical signals. If some venous pulsation is detected, the electronic device may identify the specified section as an abnormal section so as not to obtain biometric information using the plurality of optical signals. The abnormal section is a section in which the arterial pulsation and the venous pulsation are detected in some (e.g., the first optical signal and/or the second optical signal) of the plurality of optical signals, and the accuracy of biometric information (e.g., an oxygen saturation value) may be lowered due to the venous pulsation having a substantially different phase from that of the arterial pulsation. In an example, if the specified section is identified as an abnormal section, the electronic device may not obtain biometric information in the specified section.

According to an embodiment, the electronic device may repeatedly perform the operations (i.e., operations 801 to 811) in FIG. 8 in the next specified section for a period corresponding to the specified section.

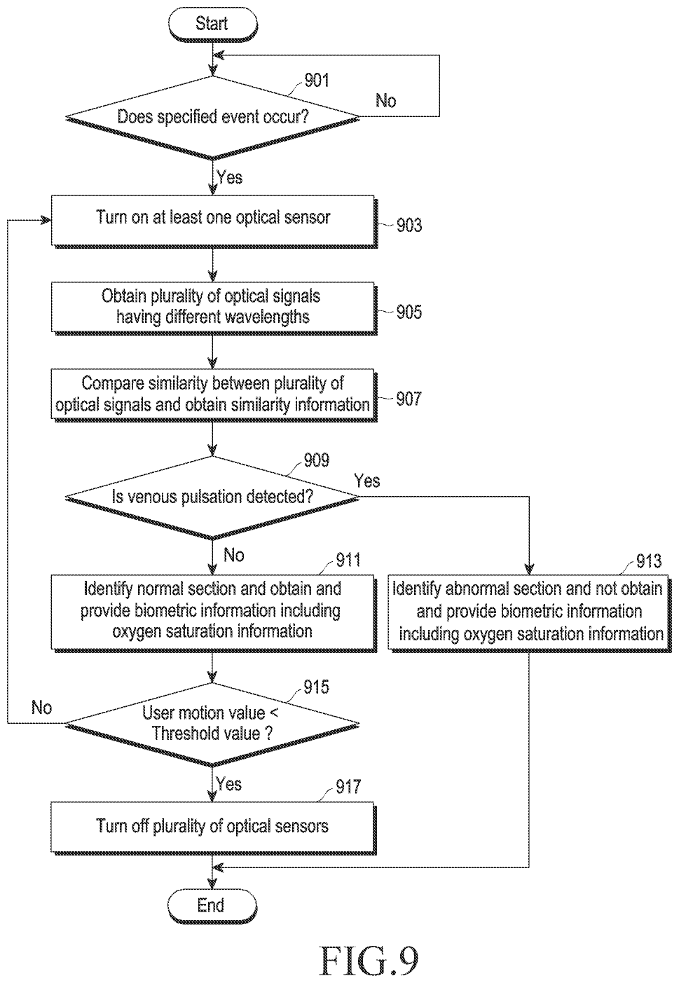

FIG. 9 is a flowchart illustrating an operation method in an electronic device according to an embodiment of the disclosure.

Although respective operations may be sequentially performed in the following embodiment, the operations are not necessarily performed sequentially. In an example, the sequence of the respective operations may vary, and at least two operations may be performed in parallel.

According to an embodiment, it may be understood that operations 901 to 917 are performed by a processor (e.g., a processor 120 in FIGS. 1 and 2) of an electronic device (e.g., an electronic device 101 in FIGS. 1 and 2).

According to an embodiment, the electronic device may intermittently perform operations in FIG. 7 at specified intervals. In an example, if obtained oxygen saturation information remains in a stable state for a specified time, at least one optical sensor may be controlled to be turned off, and then if a user's motion is detected, at least one optical sensor may be turned on and operations for obtaining biometric information described with reference to FIG. 7 may be performed.

Referring to FIG. 9, in operation 901, the electronic device according to an embodiment may identify whether or not a specified event occurs. If a specified event occurs as a result of the identification, the electronic device may perform operation 903, and if a specified event does not occur, the electronic device may perform operation 901 again. The specified event may be an event according to detection of the user's motion by at least one motion sensor in the case where the obtained oxygen saturation information remains in a stable state for a specified time.

In operation 903, the electronic device may turn on at least one optical sensor, radiate light onto a user's body part using at least one optical sensor in a specified section, and detect at least some of light reflected from the user's body part through at least one optical sensor. The specified section may be a period of sampling interval Δt. The period of sampling interval Δt (e.g., about 30 ms) may be a section including a time t1 (e.g., about a few hundred μs) for alternately turning on at least one optical sensor and a time t2 for turning off at least one optical sensor.

In operation 905, the electronic device may obtain a plurality of optical signals having different wavelengths detected by at least one optical sensor in the specified section.

In operation 907, the electronic device may obtain similarity information in which similarities between at least two optical signals among the plurality of optical signals are compared.

In operation 909, the electronic device may identify whether or not venous pulsation is detected in the specified section, based on the similarity information. As a result of the identification, if venous pulsation is not detected, the electronic device may perform operation 911, and if venous pulsation is detected, the electronic device may perform operation 913.

In operation 911, the electronic device may identify the current section as a normal section in response to non-detection of the venous pulsation and may obtain and provide biometric information including blood oxygen saturation information (an SpO2 ratio). The electronic device, for example, may display the obtained biometric information on a display (e.g., the display module 160 in FIG. 1 or the display 203 in FIG. 2) and/or transmit the same to an external electronic device (e.g., the electronic device 102, the electronic device 104, and/or the server 108 in FIG. 1) through a communication circuit (e.g., the communication module 190 in FIG. 1 or the communication circuit 205 in FIG. 2).

In operation 913, the electronic device may identify the specified section as an abnormal section in response to partial detection of the venous pulsation and may not obtain biometric information including oxygen saturation information. According to an embodiment, if low-oxygen saturation information is identified, the electronic device may ignore the low-oxygen saturation information and may not provide biometric information. According to an embodiment, if biometric information includes information related to other biometric signals in addition to the oxygen saturation information, the electronic device may provide biometric information excluding the oxygen saturation information.

In operation 915, the electronic device may identify whether or not a motion value of the user detected for a specified time is less than a threshold value. If the value is less than the threshold value as a result of the identification, the electronic device may identify that the blood oxygen saturation information remains in a stable state (e.g., a sleeping state), and the electronic device may turn off a plurality of optical sensors and terminate the operation in operation 917.

If the value is greater than or equal to the threshold value as a result of the identification in operation 915, the electronic device may identify that the user is in an active state, for example, in a motion state in which blood oxygen saturation information does not remain in the stable state, and may repeatedly perform operations 905 to 915 in the next specified section.

According to an embodiment of this document, the electronic device may compare a similarity between at least two optical signals among a plurality of optical signals having different wavelengths detected by at least one optical sensor and, if the similarity is low, identify an abnormal section in which venous pulsation is detected. Accordingly, the electronic device may obtain and provide biometric information only in the normal section by distinguishing between the abnormal section and the normal section, thereby providing biometric information (e.g., oxygen saturation information) with high accuracy. Additionally, various effects identified directly or indirectly through the disclosure may be provided.

According to an embodiment, an operation method in an electronic device (e.g., the electronic device 101 in FIGS. 1 and 2) may include radiating light onto a user's body part and receiving at least some of light reflected from the user's body part in a specified section by at least one optical sensor (the sensor module 176 in FIG. 1 or the optical sensor 201 in FIG. 2) of the electronic device.

According to an embodiment, the method may include obtaining a plurality of optical signals (e.g., the plurality of optical signals 601, 602, and 603 in FIGS. 6A to 6D) having different wavelengths from the received light in the specified section.

According to an embodiment, the method may include obtaining similarity information by comparing similarities between at least two specified optical signals among the plurality of optical signals.

According to an embodiment, the method may include identifying the specified section as an abnormal section so as not to obtain biometric information using the plurality of optical signals in response to identifying that a venous pulsation is partially detected in the specified section, based on the similarity information.

According to an embodiment, the method may include identifying the specified section as a normal section in response to identifying that the venous pulsation is not detected in the specified section, based on the similarity information, and obtaining the biometric signal in the specified section. According to an embodiment, the biometric information may include blood oxygen saturation information.

According to an embodiment, the obtaining of the similarity information may include comparing phases of at least two specified optical signals among the plurality of optical signals and identifying a similarity between the at least two specified optical signals, based on a comparison result.

According to an embodiment, the comparing of the phases may include based on identifying that the phases of the at least two specified optical signals are substantially the same, identifying that the venous pulsation is not detected from the at least two specified optical signals.

According to an embodiment, the comparing of the phases may include based on identifying that the phases of the at least two specified optical signals are different, identifying that the venous pulsation is detected from some of the at least two specified optical signals.

According to an embodiment, the comparing of the phases may include comparing the phase of at least one of a first optical signal or a second optical signal among the at least two specified optical signals with a phase of a reference signal until the time shifted to earlier or delayed by a specified time interval based on the reference signal among the at least two specified optical signals, and identifying a similarity having a maximum value among similarity values obtained by the comparing the phase of at least one of the first optical signal or the second optical signal with the phase of the reference signal.

According to an embodiment, the at least two specified optical signals may include at least one of a first optical signal and a second optical signal to be compared and a third optical signal as a reference signal, wherein the first optical signal may be a reflective photoplethysmography signal having an infrared radiation (IR) wavelength, wherein the second optical signal may be a reflective photoplethysmography signal having a red wavelength, and wherein the third optical signal may be a reflective photoplethysmography signal having a blue wavelength or green wavelength, reflected from the shallow region of the user's skin. According to an embodiment, the specified section (e.g., the sampling interval Δt in FIG. 5A) (e.g., about 30 ms) may include a time t1 (e.g., about a few hundred μs) for alternately turning on the at least one optical sensor and a time t2 for turning off the at least one optical sensor. According to an embodiment, the method may further include displaying the biometric information on a display of the electronic device and transmitting the biometric information to an external electronic device through a communication circuit of the electronic device.

According to an embodiment, the method may include obtaining the plurality of optical signals in the specified section by the at least one optical sensor based on a motion of the user detected by at least one motion sensor of the electronic device being greater than or equal to a threshold value.

According to an embodiment, the method may further include not obtaining the plurality of optical signals in the specified section by the at least one optical sensor based on the motion of the user detected by the at least one motion sensor being less than the threshold value.

According to an embodiment, a non-transitory storage medium may store a program including executable instructions configured to, when executed by a processor of an electronic device, cause the electronic device to radiate light onto a user's body part and receive at least some of light reflected from the user's body part in a specified section by at least one optical sensor of the electronic device, obtain a plurality of optical signals having different wavelengths from the received light in the specified section, obtain similarity information by comparing similarities between at least two specified optical signals among the plurality of optical signals, and identify the specified section as an abnormal section so as not to obtain biometric information using the plurality of optical signals in response to identifying that a venous pulsation is detected In the specified section, based on the similarity Information.

In addition, the embodiments disclosed in the disclosure are presented for explanation and understanding of the disclosed technical content, and are not intended to limit the scope of the technology described in the disclosure. Therefore, the scope of the disclosure should be construed to encompass all changes or various other embodiments based on the technical idea of the disclosure.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. As used herein, each of such phrases as “A or B”, “at least one of A and B”, “at least one of A or B”, “A, B, or C”, “at least one of A, B, and C”, and “at least one of A, B, or C”, may include any one of, or all possible combinations of the items enumerated together in a corresponding one of the phrases. As used herein, such terms as “1^{st}” and “2^{nd}”, or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic”, “logic block”, “part”, or “circuitry”. A module, for example, may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it. This allows the machine to be operated to perform at least one function according to the at least one instruction invoked. The one or more instructions may include a code generated by a complier or a code executable by an interpreter. The machine-readable storage medium, for example, may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to some embodiments, each component (e.g., a module or a program) of the above-described components may include a single entity or multiple entities, and some of the multiple entities may be separately disposed in different components. According to other embodiments, one or more of the above-described components or operations may be omitted, or one or more other components or operations may be added. Alternatively or additionally, a plurality of components (e.g., modules or programs) may be integrated into a single component. In such a case, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
at least one optical sensor;
a memory; and
at least one processor electrically connected to the at least one optical sensor and the memory,
wherein the at least one processor is configured to:
control the at least one optical sensor to radiate light onto a user's body part and to receive at least some of light reflected from the user's body part in a specified section,
obtain a plurality of optical signals having different wavelengths from the received light in the specified section,
obtain similarity information by comparing similarities between at least two specified optical signals among the plurality of optical signals, and
identify the specified section as an abnormal section so as not to obtain biometric information using the plurality of optical signals in response to identifying that a venous pulsation is detected in the specified section, based on the similarity information.

2. The electronic device of claim 1, wherein the at least one processor is further configured to not obtain the biometric information in the specified section in response to identifying the specified section as an abnormal section.

3. The electronic device of claim 1,
wherein the at least one processor is further configured to identify the specified section as a normal section in response to identifying that the venous pulsation is not detected in the specified section, based on the similarity information, and obtain the biometric information in the specified section, and
wherein the biometric information comprises blood oxygen saturation information.

4. The electronic device of claim 1,
wherein the at least one processor is further configured to:
compare phases of the at least two specified optical signals among the plurality of optical signals, and
identify a similarity between the at least two specified optical signals, based on a result of the comparison, and
wherein the at least two specified optical signals are at least one of a first optical signal or a second optical signal among the plurality of optical signals, and a third optical signal among the plurality of optical signals, which is a reference signal.

5. The electronic device of claim 4, wherein the at least one processor is further configured to:
based on identifying that the phases of the at least two specified optical signals are same, identify that the venous pulsation is not detected from the at least two specified optical signals.

6. The electronic device of claim 4, wherein the at least one processor is further configured to:
based on identifying that the phases of the at least two specified optical signals are different, identify that the venous pulsation is detected from some of the at least two specified optical signals.

7. The electronic device of claim 4, wherein the at least one processor is further configured to:
compare the phase of at least one of the first optical signal or the second optical signal among the at least two specified optical signals with a phase of the reference signal until a time shifted to earlier or delayed by a specified time interval based on the reference signal among the at least two specified optical signals, and
identify a similarity having a maximum value among similarity values obtained by the comparing the phase of at least one of the first optical signal or the second optical signal with the phase of the reference signal.

8. The electronic device of claim 1,
wherein the at least two specified optical signals comprise at least one of a first optical signal and a second optical signal to be compared and a third optical signal as a reference signal,
wherein the first optical signal is a reflective photoplethysmography signal having an infrared radiation (IR) wavelength,
wherein the second optical signal is a reflective photoplethysmography signal having a red wavelength,
wherein the third optical signal is a reflective photoplethysmography signal having a blue wavelength or green wavelength, reflected from a shallow region of a user's skin, and
wherein the specified section comprises a time (t1) for turning on the at least one optical sensor and a time (t2) for turning off the at least one optical sensor.

9. The electronic device of claim 1, further comprising:
a display electrically connected to the at least one processor; and
a communication circuit electrically connected to the at least one processor,
wherein the at least one processor is configured to control the display to display the biometric information and control the communication circuit to transmit the biometric information to an external electronic device.

10. The electronic device of claim 1, wherein the at least one processor is further configured to:
control the at least one optical sensor to obtain the plurality of optical signals in the specified section based on a motion of the user detected by at least one motion sensor being greater than or equal to a threshold value, and
control the at least one optical sensor not to obtain the plurality of optical signals in the specified section based on the motion of the user detected by the at least one motion sensor being less than the threshold value.

11. An operation method in an electronic device, the method comprising:
radiating light onto a user's body part and receiving at least some of light reflected from the user's body part in a specified section by at least one optical sensor of the electronic device;
obtaining a plurality of optical signals having different wavelengths from the received light in the specified section;
obtaining similarity information by comparing similarities between at least two specified optical signals among the plurality of optical signals; and
identifying the specified section as an abnormal section so as not to obtain biometric information using the plurality of optical signals in response to identifying that a venous pulsation is detected in the specified section, based on the similarity information.

12. The method of claim 11, comprising:
identifying the specified section as a normal section in response to identifying that the venous pulsation is not detected in the specified section, based on the similarity information; and
obtaining the biometric information in the specified section,
wherein the biometric information comprises blood oxygen saturation information.

13. The method of claim 11, wherein the obtaining of the similarity information comprises:
comparing phases of at least two specified optical signals among the plurality of optical signals; and
identifying a similarity between the at least two specified optical signals, based on a result of the comparison.

14. The method of claim 13, wherein the comparing of the phases comprises:
based on identifying that the phases of the at least two specified optical signals are the same, identifying that the venous pulsation is not detected from the at least two specified optical signals.

15. The method of claim 13, wherein the comparing of the phases comprises:
based on identifying that the phases of the at least two specified optical signals are substantially different, identifying that the venous pulsation is detected from some of the at least two specified optical signals.

16. The method of claim 13, wherein the comparing of the phases comprises:
comparing the phase of at least one of a first optical signal or a second optical signal among the at least two specified optical signals with a phase of a reference signal until a time shifted to earlier or delayed by a specified time interval based on the reference signal among the at least two specified optical signals; and
identifying a similarity having a maximum value among similarity values obtained by the comparing the phase of at least one of the first optical signal or the second optical signal with the phase of the reference signal.

17. The method of claim 11,
wherein the at least two specified optical signals comprise at least one of a first optical signal and a second optical signal to be compared and a third optical signal as a reference signal,
wherein the first optical signal is a reflective photoplethysmography signal having an infrared radiation (IR) wavelength,
wherein the second optical signal is a reflective photoplethysmography signal having a red wavelength,
wherein the third optical signal is a reflective photoplethysmography signal having a blue wavelength or green wavelength, reflected from a shallow region of a user's skin, and
wherein the specified section comprises a time (t1) for turning on the at least one optical sensor and a time (t2) for turning off the at least one optical sensor.

18. The method of claim 11, further comprising:
displaying the biometric information on a display of the electronic device; and
transmitting the biometric information to an external electronic device through a communication circuit of the electronic device.

19. The method of claim 11, further comprising:
obtaining the plurality of optical signals in the specified section by the at least one optical sensor based on a motion of the user detected by at least one motion sensor of the electronic device being greater than or equal to a threshold value; and
not obtaining the plurality of optical signals in the specified section by the at least one optical sensor based on the motion of the user detected by the at least one motion sensor being less than the threshold value.

20. A non-transitory storage medium storing a program comprising executable instructions configured to, when executed by a processor of an electronic device, cause the electronic device to:
radiate light onto a user's body part and receive at least some of light reflected from the user's body part in a specified section by at least one optical sensor of the electronic device;
obtain a plurality of optical signals having different wavelengths from the received light in the specified section;
obtain similarity information by comparing similarities between at least two specified optical signals among the plurality of optical signals; and
identify the specified section as an abnormal section so as not to obtain biometric information using the plurality of optical signals in response to identifying that a venous pulsation is detected in the specified section, based on the similarity information.

21. The non-transitory storage medium of claim 20, wherein the venous pulsation includes significant blood flow in a venous bed under a user's skin.

22. The non-transitory storage medium of claim 20, wherein the abnormal section includes a section in which an arterial pulsation and the venous pulsation are detected in the plurality of optical signals.
